(19) United States
(12) Patent Application Publication
(10) Pub. No.: US 2025/0275673 A1
(43) Pub. Date: Sep. 4, 2025

(54) MAGNETIC ANCHORED AND ACTUATED SYSTEM AND MANUFACTURING METHOD THEREOF

(71) Applicant: MULTI-SCALE MEDICAL ROBOTICS CENTER LIMITED, Hong Kong (CN)

(72) Inventors: Zheng LI, Hong Kong (CN); Jixiu LI, Hong Kong (CN); Tao ZHANG, Hong Kong (CN); TRUMAN CHENG, Hong Kong (CN); Sze Hang Calvin NG, Hong Kong (CN); Wai Yan Philip CHIU, Hong Kong (CN)

(21) Appl. No.: 18/857,923

(22) PCT Filed: Apr. 20, 2023
(86) PCT No.: PCT/IB2023/054019

§ 371 Date: Oct. 18, 2024

Related U.S. Application Data
(60) Provisional application No. 63/333,099, filed on Apr. 20, 2022.

(51) Int. Cl. A61B 1/00

(52) U.S. Cl. CPC A61B 1/00158; A61B 1/00057; A61B 1/0011

(57) ABSTRACT

This invention provides a magnetic anchored and actuated system and manufacturing method thereof. In one embodiment, said system comprises: (a) an internal unit for insertion into a patient's body, comprising an internal frame, an internal magnetic rotator, and a function module; and (b) an external unit for anchoring and controlling locomotion of said internal unit, comprising an external frame, an external magnetic rotator, and an actuation module.

### FIELD OF THE INVENTION

This invention relates to magnetic anchored and actuated systems.

### BACKGROUND OF THE INVENTION

Minimally invasive surgery (MIS) has been widely applied in the modern medical field with the clinical benefits of shorter convalescence, less postoperative pain and improved cosmesis. Magnetic anchored and actuated systems have high potential in clinical applications, such as assistive devices for MIS, like the magnetic endoscope systems proposed by Cadeddu et al. and Valdastri et al. However, the anchoring distance in these systems is short and cannot cover the body cavity thickness. To increase the effective anchoring and actuation distance, larger magnets are required, which complex the system. Magnetic anchored and actuated systems have been developed in research labs aiming for MIS, such as laparoscopic surgery and thoracic surgery. However, none has been adopted clinically. One of the major issues is that the effective magnetic anchoring distance cannot cover the full thickness of the body cavities. Existing methods can provide an anchoring distance that ranges from 20-50 mm, just like the system reported by Liu et al. and Cheng et al, which uses big magnets to anchor and actuate a capsule-shaped endoscope in the thoracic or abdominal cavity. On the contrary, wall thickness for the chest cavity can reach 80 mm or more. Larger magnets can be used to increase the anchoring and actuation distance. However, this will make the system bulky and oversize. The consequence is that the device may not be able to insert into the chest cavity through the intercostal space, or a larger incision port is needed.

This invention provides a magnet configuration that could effectively increase the anchoring and actuation distance while keeping the system design compact. Many devices in MIS face the problem of space constraints, such as endoscope, tissue retractor, organ clamp, blood sucker and so on. This configuration can also provide a large working range for surgeons to operate those instruments, making the whole surgery more convenient and reliable. The magnet configuration can realize the benefits of large anchoring distance and compact dimension, making the magnetic actuation instruments can be applied in the actual application in uniport VATS (Video-assisted thoracoscopic surgery). Compared with the traditional MIS devices, they can alleviate the problems of port crowding and instruments fencing for surgeons and relieve the burden of holding and operating these devices by the assistant.

Information that was published before the filing of this disclosure includes those listed under the Reference List.

### SUMMARY OF THE INVENTION

This invention provides a magnetic anchored and actuated system. In one embodiment, said magnetic anchored and actuated system comprises: a) An internal unit for insertion into a patient's body, comprising an internal frame, an internal magnetic rotator, and a function module; and b) An external unit for anchoring and controlling locomotion of said internal unit, comprising an external frame, an external magnetic rotator, and an actuation module; wherein, said internal magnetic rotator comprises an internal shell, a left internal magnetic component, a middle internal magnetic component, and a right internal magnetic component; said external magnetic rotator comprises an external shell, a left external magnetic component, a middle external magnetic component, and a right external magnetic component; said left external magnetic component and said left internal magnetic component are radially magnetized with magnetization direction of said left external magnetic component being same as said left internal magnetic component; said right external magnetic component and said right internal magnetic component are radially magnetized with magnetization direction of said right external magnetic component being same as said right internal magnetic component; each of said left external magnetic component and said left internal magnetic component has a magnetization direction opposite to each of said right external magnetic component and right internal magnetic component; said middle external magnetic component and said middle internal magnetic component are axially magnetized with magnetization direction of said middle external magnetic component being opposite to said middle internal magnetic component; and each of said left internal magnetic component, middle internal magnetic component and right internal magnetic component is magnetically coupled to each of said left external magnetic component, middle external magnetic component, and right external magnetic component respectively; said actuation module controls motion of said external magnetic rotator, said motion comprises rotation and translation; and said function module provides one or more specific functionalities along with movement of said internal magnetic rotator.

A method for assembling the external magnetic rotator of this invention is further provided. In one embodiment, said method comprises the steps of: a) providing a shell body made of non-ferromagnetic materials; b) Placing the middle external magnetic component in middle part of the shell body; c) Placing and fixing a middle plate; d) Inserting two magnetic shielding plates on both sides of the middle external magnetic component to shield magnetic field; e) Installing the left external magnetic component and the right external magnetic component; f) Placing and fixing a right plate and a left plate, wherein said left plate and right plate are made of non-ferromagnetic materials; and g) Remove the two magnetic shielding plates.

### BRIEF DESCRIPTION OF THE FIGURES

FIG. 1A and FIG. 1B show the overview of the whole magnetic system and simulated clinical application scenario. These include the external holding device 1, external unit 2 and internal unit 3. The internal unit is inserted into the patient and anchored on the inner wall of the chest wall 4 thorough the MIS incision 4-1.

FIGS. 2A and 2B show the structure of the external unit 2 in an embodiment of this invention, which is composed of an external magnetic rotator 2-1, an external frame 2-2 and an actuation module 2-3. The actuation module 2-3 is composed of a motor 2-3-1, an upper pulley 2-3-2, a synchronous belt 2-3-3 and a lower pulley 2-3-4.

FIG. 3A and FIG. 3B show the structure of the external magnet rotator 2-1 in an embodiment of this invention. In this embodiment, the external magnet rotator 2-1 further comprises the LEPM 2-1-1, the MEPM 2-1-2 and the REPM 2-1-3 that are held by the left plate 2-1-4, rotator shell 2-1-5, middle plate 2-1-6 and right plate 2-1-7.

FIG. 4A and FIG. 4B show the structure of the external frame 2-2 in an embodiment of this invention. In this embodiment, the external frame 2-2 further comprises upper frame 2-2-1, the Permalloy plate on the frame 2-2-2, the lower left frame 2-2-3 and the lower right frame 2-2-4.

FIG. 5A and FIG. 5B show the structure of the internal unit 3 in an embodiment of this invention, which is composed of an internal magnetic rotator 3-1, a function module 3-2 and an internal frame 3-3. The internal magnet rotator 3-1 further comprises the LIPM 3-1-1, the MIPM 3-1-2 and the RIPM 3-1-3 that are held by left shell 3-1-4, the middle shell 3-1-5 and the right shell 3-1-6. The internal frame 3-3 further comprise the internal left frame 3-3-1 and internal right frame 3-3-2.

FIG. 6 shows another approach to place the function module in an embodiment of this invention. In this embodiment, the function module 3-2 is attached on a pneumatic soft bello

FIG. 7 shows another approach to place the function module in an embodiment of this invention. In this embodiment, the function module 3-2 is attached on a rigid link I 3-1-10, a joint 3-1-11 and a rigid link II 3-1-12.

FIG. 8 shows another approach to place the function module in an embodiment of this invention. In this embodiment, the function module 3-2 is attached on a flexible link 3-1-13.

FIG. 9 shows the magnet distribution and configuration of the magnetic system in an embodiment of this invention, the magnets include LEPM 2-1-1, MEPM 2-1-2, REPM 2-1-3, LIPM 3-1-1, MIPM 3-1-2 and RIPM 3-1-3.

FIG. 10A to FIG. 10H show the magnetic field distribution of this invention and another magnet configuration.

FIG. 11A and FIG. 11B shows the comparison results of the anchoring performance among this invention and some other magnets configuration. (A) is the curve reflecting the relationship between anchoring force (Fz) and distance; (B) is the curve reflecting the relationship between attractive force per unit volume (FPV) and distance.

FIG. 12A to FIG. 12L shows the assembly process of the external magnetic rotator in an embodiment of this invention comprising seven steps.

### DETAILED DESCRIPTION OF THE INVENTION

Magnetic forces provide non-contact motion transmission. This enables the development of magnetic anchored and actuated devices that could be placed inside a body cavity and actuated outside the body. With a direct magnetic coupling, the anchoring and effective actuation range is limited due to the exponential attenuation of magnetic field strength. This patent discloses a magnet coupling method that could effectively increase the anchoring and actuation range. With this method, design examples for minimally invasive surgery are also provided.

The magnetic system is composed of two parts, an internal unit that is inside the patient body and an external unit that is outside the patient body. Both the internal unit and the external unit contain magnetic components. The external unit is held and actuated by an external holding device to anchor the internal unit to the patient's inner wall of the body cavity and drive the internal unit to move and rotate with the help of the magnetic coupling.

The external unit is composed of an external magnetic rotator, an external frame and an actuation module. The external magnetic rotator consists of an axially magnetized external permanent magnets (EPM) in the middle part (MEPM) and two radially magnetized EPMs at left and right ends (LEPM and REPM). All the EPMs are fixed on the shell of the external magnetic rotator. The external magnetic rotator is connected to an external frame and can rotate around its central axis with the help of the actuation module. The internal unit is composed of an internal magnetic rotator, a function module and an internal frame. The internal magnetic rotator consists of an axially magnetized internal permanent magnets (IPM) in the middle part (MIPM) and two radially magnetized IPMs at left and right ends (LIPM and RIPM). All the IPMs are fixed on the shell of the internal magnetic rotator. A function module integrating camera, inertial measurement unit (IMU), LEDs and other components can provide the image, orientation feedback, lighting and other information or condition during the surgery. The internal magnetic rotator is connected to an internal frame in some way and can rotate around its central axis.

This invention provides a magnetic anchored and actuated system. In one embodiment, said magnetic anchored and actuated system comprises: a) An internal unit for insertion into a patient's body, comprising an internal frame, an internal magnetic rotator, and a function module; and b) An external unit for anchoring and controlling locomotion of said internal unit, comprising an external frame, an external magnetic rotator, and an actuation module; wherein, said internal magnetic rotator comprises an internal shell, a left internal magnetic component, a middle internal magnetic component, and a right internal magnetic component; said external magnetic rotator comprises an external shell, a left external magnetic component, a middle external magnetic component, and a right external magnetic component; said left external magnetic component and said left internal magnetic component are radially magnetized with magnetization direction of said left external magnetic component being same as said left internal magnetic component; said right external magnetic component and said right internal magnetic component are radially magnetized with magnetization direction of said right external magnetic component being same as said right internal magnetic component; each of said left external magnetic component and said left internal magnetic component has a magnetization direction opposite to each of said right external magnetic component and right internal magnetic component; said middle external magnetic component and said middle internal magnetic component are axially magnetized with magnetization direction of said middle external magnetic component being opposite to said middle internal magnetic component; and each of said left internal magnetic component, middle internal magnetic component and right internal magnetic component is magnetically coupled to each of said left external magnetic component, middle external magnetic component, and right external magnetic component respectively; said actuation module controls motion of said external magnetic rotator, said motion comprises rotation and translation; and said function module provides one or more specific functionalities along with movement of said internal magnetic rotator.

In one embodiment, said left external magnetic component, middle external magnetic component, right external magnetic component, left internal magnetic component, middle internal magnetic component or right internal magnetic component comprises one or more magnets. In another embodiment, said one or more magnets are selected from the group consisting of permanent magnets, electromagnets and soft magnetic materials.

In one embodiment, said left internal magnetic component, right internal magnetic component, left external magnetic component, right external magnetic component, middle internal magnetic component or middle external magnetic component has a shape selected from the group consisting of cylindrical, semi-cylindrical, cuboid, sphere, semi-sphere, and ellipsoid.

In one embodiment, said internal shell holds in place said left internal magnetic component, said middle internal magnetic component and said right internal magnetic component.

In one embodiment, said internal magnetic rotator is connected via a first connection mechanism to said internal frame for rotation around a central axis. In one embodiment, said first connection mechanism comprises one or more selected from the group consisting of bearings, shaft, compliant structure, and soft structure.

In one embodiment, said one or more specific functionalities comprises providing one or more functions selected the group consisting of lighting, imaging, tissue/organ retraction, instrument manipulation, and fluid suction.

In one embodiment, said function module is attached to the internal magnetic rotator by one or more methods selected from the group consisting of glue, movable joints, and compliant/soft structures.

In one embodiment, said external shell comprises a shell body, a left plate, a middle plate and a right plate for holding said left external magnetic component, middle external magnetic component and right external magnetic component respectively; wherein said shell body, left plate and right plate are made of non-ferromagnetic materials.

In one embodiment, said external magnetic rotator further comprises one or more onboard sensors for measuring location and motion. In another embodiment, said one or more onboard sensors are selected from the group consisting of inertial measurement unit, gyroscope, accelerator, hall sensor, encoder, and optical marker.

In one embodiment, said external magnetic rotator is connected via a second connection mechanism to said external frame for rotation around a central axis. In another embodiment, said second connection mechanism comprises one or more selected from the group consisting of bearings, shaft, compliant structure, and soft structure.

In one embodiment, said actuation module comprises an actuator for rotating said external magnetic rotator and an external holding device for spatially moving said external magnetic rotator. In another embodiment, said actuator comprises a motor. In a further embodiment, said external holding device comprises a robot arm.

A method for assembling the external magnetic rotator of this invention is further provided. In one embodiment, said method comprises the steps of: a) providing a shell body made of non-ferromagnetic materials; b) Placing the middle external magnetic component in middle part of the shell body; c) Placing and fixing a middle plate; d) Inserting two magnetic shielding plates on both sides of the middle external magnetic component to shield magnetic field; e) Installing the left external magnetic component and the right external magnetic component; f) Placing and fixing a right plate and a left plate, wherein said left plate and right plate are made of non-ferromagnetic materials; and g) Remove the two magnetic shielding plates.

In one embodiment, said magnetic shielding plates are made of permalloy.

The invention will be better understood by reference to the Experimental Details which follow, but those skilled in the art will readily appreciate that the specific experiments described are only for illustrative purposes and are not meant to limit the invention as described herein, which is defined by the claims that follow thereafter.

Throughout this application, various references or publications are cited. Disclosures of these references or publications in their entireties are hereby incorporated by reference into this application in order to more fully describe the state of the art to which this invention pertains. It is to be noted that the transitional term “comprising”, which is synonymous with “including”, “containing” or “characterized by”, is inclusive or open-ended and does not exclude additional, un-recited elements or method steps.

### Details of the Invention Operation/Functions

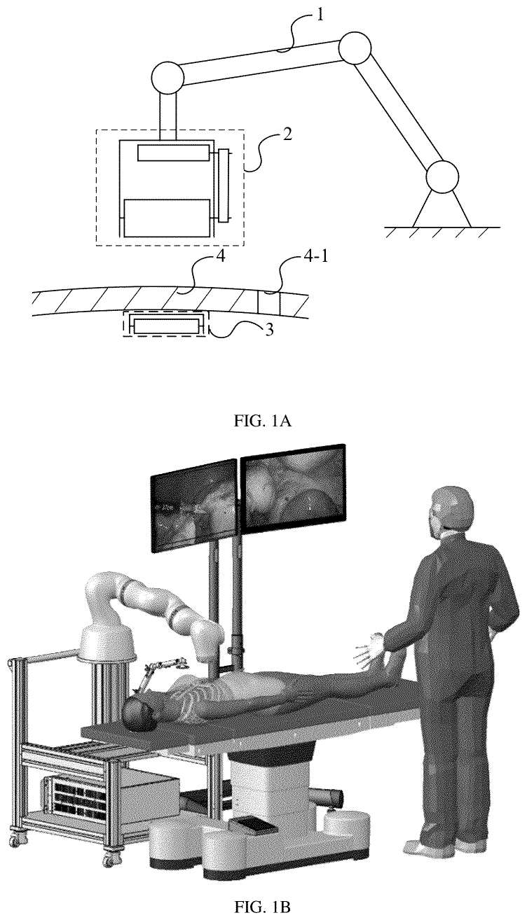

FIG. 1A and FIG. 1B show the application scenario of this magnetic system in MIS. At the beginning of the surgery, the internal unit 3 will be inserted into the patient's body via the MIS incision 4-1 and be anchored on the patient's inner wall of the cavity by the attractive force generated from the external unit 2. Next, the external unit 2 held by the external holding device 1 will move to drag the internal unit 3 to the desired position. As the surgical procedure progresses, the external unit 2 can guide the internal unit 3 to translate and rotate to perform corresponding motions to fulfill the surgeon's needs. Finally, the internal unit 3 will move to the vicinity of the MIS incision 4-1 and be taken out.

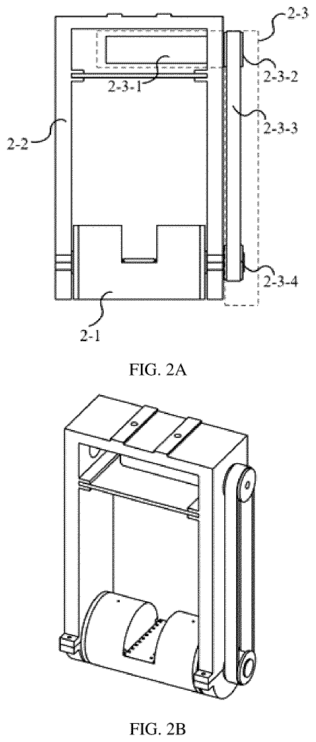

FIG. 2A and FIG. 2B are an illustration of the external unit 2 which is further composed of the external magnetic rotator 2-1, an external frame 2-2 and an actuation module 2-3. In this actuation module 2-3, the motor 2-3-1 is fixed on the external frame 2-2. The upper pulley 2-3-2 is fixed on the motor 2-3-1 and connected with the lower pulley 2-3-4 with a synchronous belt 2-3-3. The lower pulley is fixed on the magnetic rotator 2-1. So, when the motor 2-3-1 is working, it can actuate the magnetic rotator to rotate.

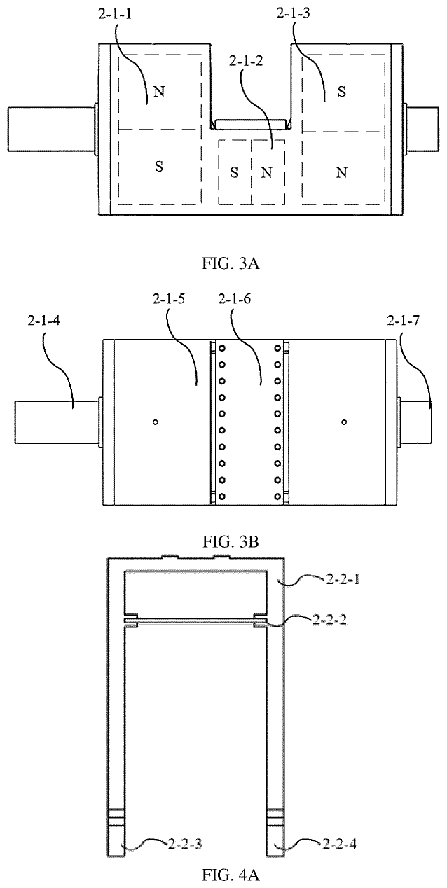

FIG. 3A and FIG. 3B show the composition of the magnetic rotator 2-1. It has three magnets. The magnet on the left side (LEPM 2-1-1) has the magnetization direction from bottom to the top. The magnet on the middle part (MEPM 2-1-2) has the magnetization direction from left to the right. The magnet on the right side (REPM 2-1-3) has the magnetization direction from top to the bottom. All the magnets are placed on the rotator shell 2-1-5. A left plate 2-1-4 is connected to the rotator shell 2-1-5 to hold the LEPM 2-1-1. A middle plate 2-1-6 is connected to the rotator shell 2-1-5 to hold the MEPM 2-1-2. A right plate 2-1-7 is connected to the rotator shell 2-1-5 to hold the REPM 2-1-3.

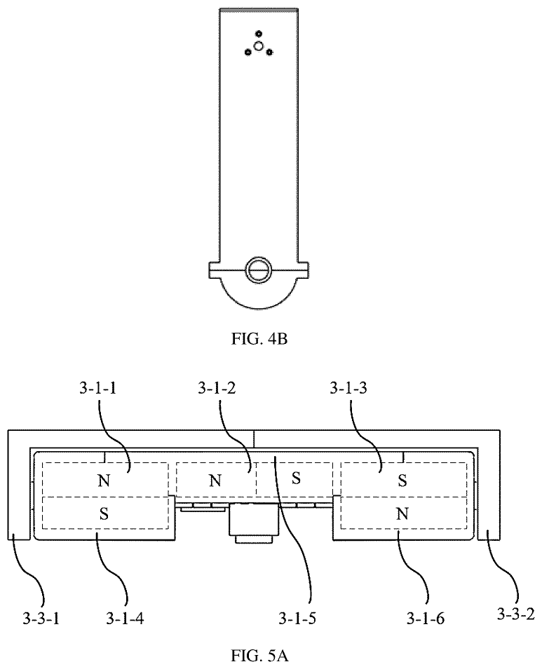

FIG. 4A and FIG. 4B give one example of the external frame. The upper frame 2-2-1 holds the motor 2-3-1 to actuate the magnetic rotator 2-1 and a Permalloy plate on the frame 2-2-2 to shield the effect of magnetic fields on the motor 2-3-1. The lower left frame 2-2-3 and the lower right frame 2-2-4 is connected on the upper frame 2-2-1 to hold the magnetic rotator 2-1.

FIG. 5A and FIG. 5B give one example of the internal unit 3. It is composed of an internal magnetic rotator 3-1, a function module 3-2 and an internal frame 3-3. The internal magnetic rotator 3-1 has three magnets. The magnet on the left side (LIPM 3-1-1) has the magnetization direction from bottom to the top. The magnet on the middle part (MIPM 3-1-2) has the magnetization direction from left to the right. The magnet on the right side (REPM 3-1-3) has the magnetization direction from top to the bottom. A left shell 3-1-4 is used to hold the LIPM 3-1-1. A middle shell 3-1-5 is used to hold the LIPM 3-1-2. A right shell 3-1-6 is used to hold the RIPM 3-1-6. The internal left frame 3-3-1 and internal right frame 3-3-2 are connected to hold the internal magnetic rotator 3-1 that can rotate around its own central axis. The function module 3-2 is placed on the lower half of the middle part. It can provide the image, orientation feedback, lighting and other information or condition during the surgery.

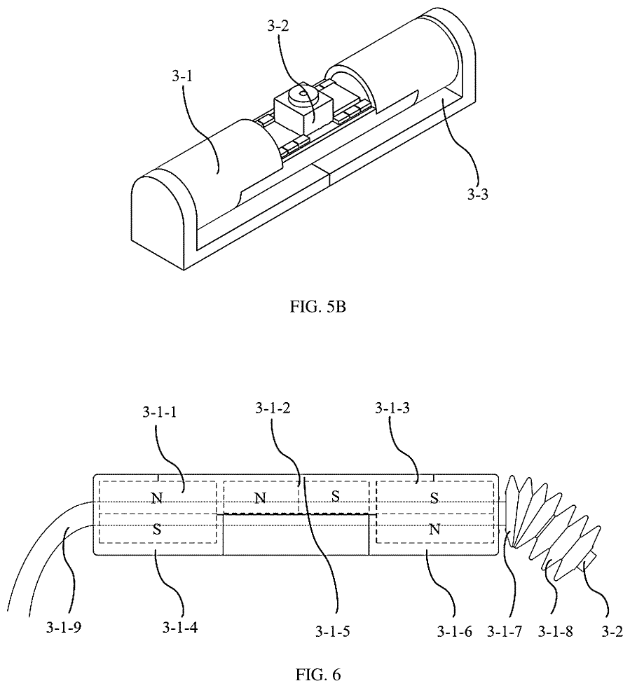

FIG. 6 gives another example of the internal unit 3. The magnet on the left side (LIPM 3-1-1) has the magnetization direction from bottom to the top. The magnet on the middle part (MIPM 3-1-2) has the magnetization direction from left to the right. The magnet on the right side (REPM 3-1-3) has the magnetization direction from top to the bottom. A left shell 3-1-4 is used to hold the LIPM 3-1-1. A middle shell 3-1-5 is used to hold the LIPM 3-1-2. A right shell 3-1-6 is used to hold the RIPM 3-1-6. The pneumatic soft bellow I 3-1-7 which can extend the length is attached on the torque coil 3-1-9. The torque coil 3-1-9 can rotate to provide a rotational degree of freedom. The pneumatic soft bellow II 3-1-8 which can bend is attached on the pneumatic soft bellow 1 3-1-7. And the function module 3-2 is attached on the pneumatic soft bellow II 3-1-8.

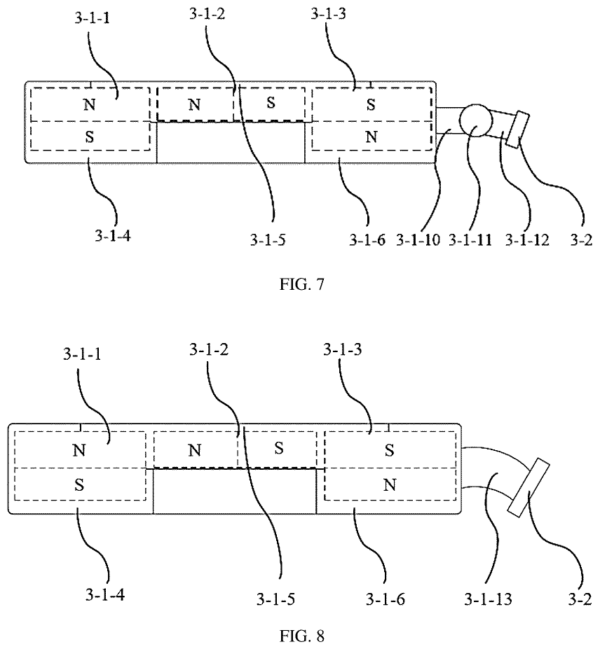

FIG. 7 gives another example of the internal unit 3. The magnet on the left side (LIPM 3-1-1) has the magnetization direction from bottom to the top. The magnet on the middle part (MIPM 3-1-2) has the magnetization direction from left to the right. The magnet on the right side (REPM 3-1-3) has the magnetization direction from top to the bottom. A left shell 3-1-4 is used to hold the LIPM 3-1-1. A middle shell 3-1-5 is used to hold the LIPM 3-1-2. A right shell 3-1-6 is used to hold the RIPM 3-1-6. The rigid link I 3-1-10 is attached on the right shell 3-1-6. The joint 3-1-11 which can be actuated by magnetic field or motor is attached on the rigid link I 3-1-10. The rigid link II 3-1-12 is attached on the joint 3-1-11. And the function module 3-2 is attached on the rigid link II 3-1-12.

FIG. 8 gives another example of the internal unit 3. The magnet on the left side (LIPM 3-1-1) has the magnetization direction from bottom to the top. The magnet on the middle part (MIPM 3-1-2) has the magnetization direction from left to the right. The magnet on the right side (REPM 3-1-3) has the magnetization direction from top to the bottom. A left shell 3-1-4 is used to hold the LIPM 3-1-1. A middle shell 3-1-5 is used to hold the LIPM 3-1-2. A right shell 3-1-6 is used to hold the RIPM 3-1-6. The flexible link 3-1-13 is attached on the right shell 3-1-6. The flexible link 3-1-13 can be soft magnetic material, cable-driven flexible structure and so on. And the function module 3-2 is attached on the flexible link 3-1-13.

Of course, for the function module, here are many other variations based on this magnet configuration, such as a tissue retractor, organ clamp, blood sucker and so on. They can also be inserted into the patient body and anchored on the inner wall of the body cavity to preform different functions. This design can also enhance their anchoring distance and working stability.

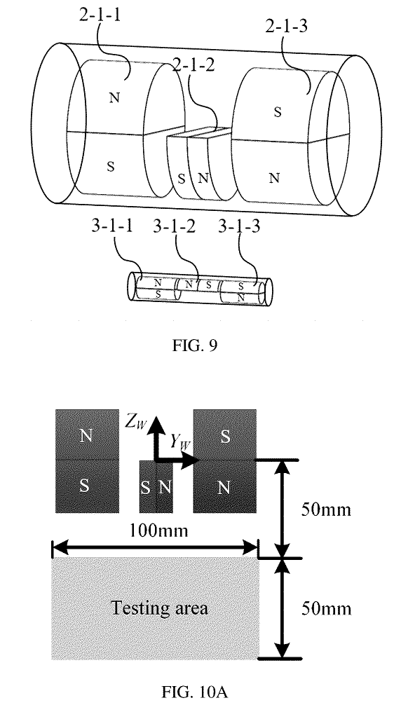

FIG. 9 show the magnet distribution and configuration of the magnetic system. the magnetization direction of LEPM 2-1-1 and LIPM 2-1-3 is opposite to that of REPM 3-1-1 and RIPM 3-1-3, leading to the repulsive force between LEPM 2-1-1 and RIPM 3-1-3 and repulsive force between REPM 2-1-3 and LIPM 3-1-1. This effect will significantly weaken the attractive force applied to the internal unit, resulting in a shorter anchoring distance between the bottom of EPMs and top of IPMs. Therefore, MEPM 2-1-2 and MIPM 3-1-2 are introduced to improve the anchoring performance of the capsule. The magnetic coupling between them can generate extra attractive force to increase the working range. In addition, MEPM 2-1-2 can also attract LIPM 3-1-1 and RIPM 3-1-3, which is the same as MIPM 3-1-2 can be attracted by LEPM 2-1-1 and REPM 2-1-3, providing additional anchoring force.

As for the design optimization for this magnet configuration, it is mainly about the size, shape, and strength of the magnets and the distance between the magnets caged in one rotator. These parameters can be determined by the demand of working distance and motion range. The performance of the system can be evaluated by theoretical analysis and finite element analysis. Also, the difficulty of installation, process of fabrication and cost should also be taken into consideration.

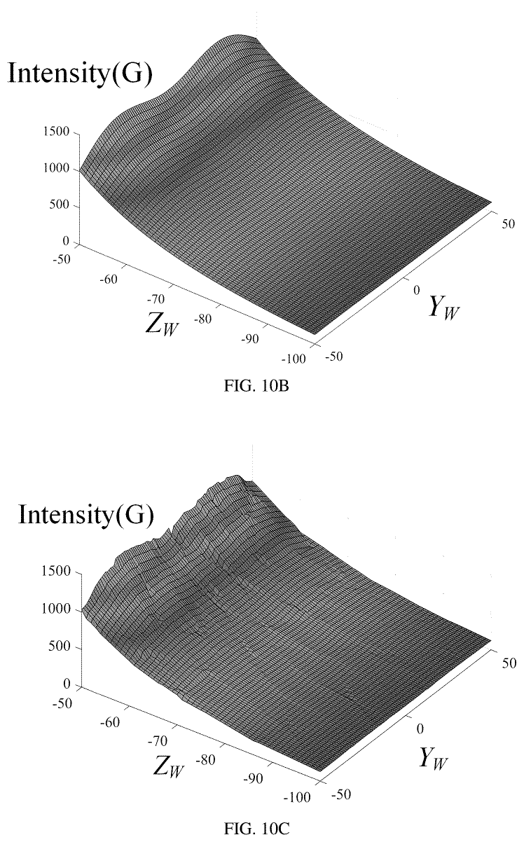

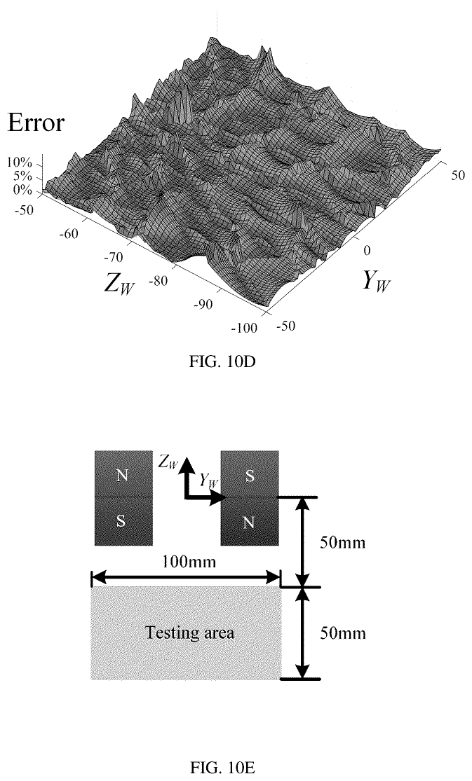

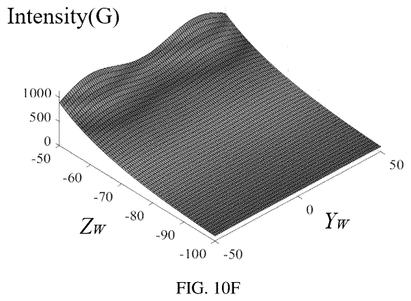

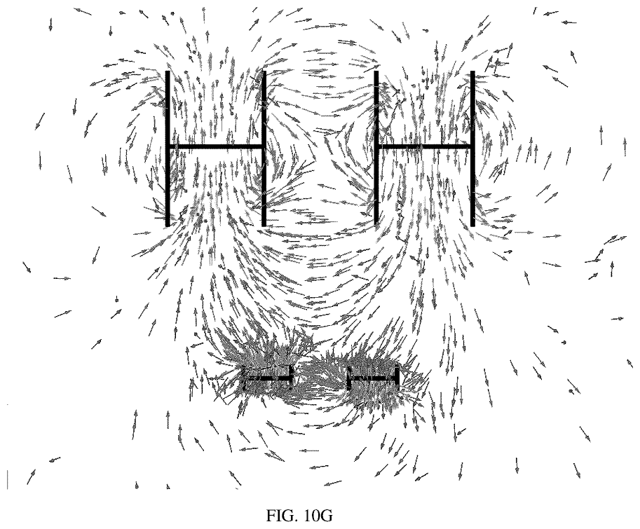

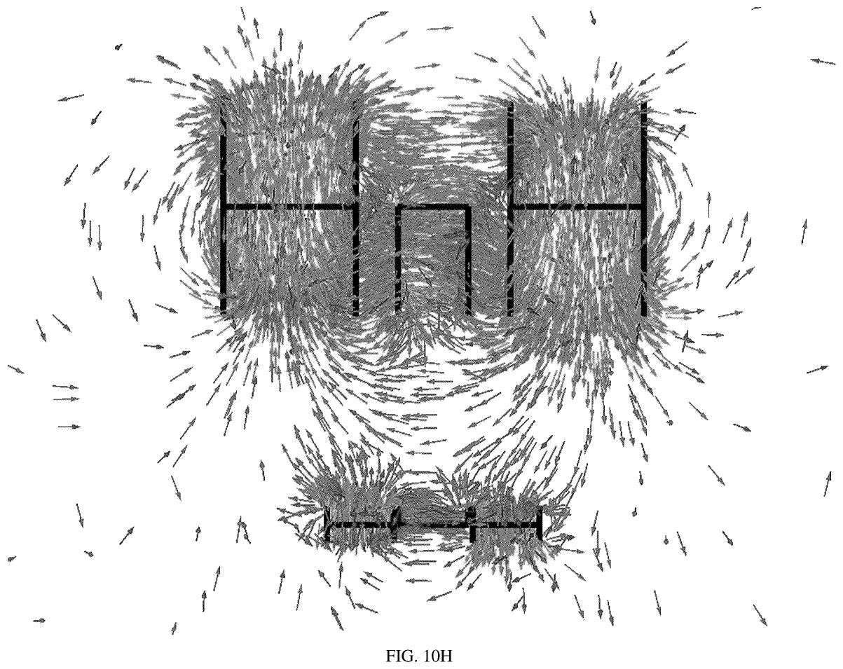

FIG. 10A to FIG. 10H show the magnetic field distribution of different magnet configurations. In YZ plane, one testing area below the EPMs was selected for evaluation. It had a size of 100 mm×50 mm, and the sampling interval was set as 1 mm (as shown in FIG. 10A). Then, the norm of magnetic field intensity distributed in that region was obtained by model calculation. Here the magnetic dipole model is adopted to get the magnetic field situation, the magnetic field strength generated by the a^{th} EPM at the b^{th} location is obtained by:

$${B}_{a b} = \frac{{μ}_{0}}{4 π {‖ {P}_{l b} - {P}_{E a} ‖}^{3}} ( \frac{3 ( {P}_{l b} - {P}_{E a} ) {( {P}_{l b} - {P}_{E a} )}^{T}}{{‖ {P}_{l b} - {P}_{E a} ‖}^{2}} - l ) {R}_{E} {M}_{E a}$$

where μ₀ is the vacuum permeability that is a constant; I∈R^{3×3} is an identity matrix; M_{Ea} =[m_{Eax}, m_{Eay}, M_{Eaz}]^{T} ∈ [symbol] ^{3×1} indicates the magnetic moment vector of the a^{th} EPM at initial position; R_{E}∈ [symbol] ^{3×3} is the rotation matrix of the external magnetic rotator. Then superpose the magnetic field of all the EPMs and calculate the norm of the magnetic field to get the result of the FIG. 10B. The finite element analysis is also performed to verify the theoretical model. The result is shown in FIG. 10C and the error situation is shown in FIG. 10D. It can be seen that the result of model of and simulation can math very well. Another magnet configuration is picked to compare the magnetic field distribution that is shown in FIG. 10E and FIG. 10F. And the overall magnetic field distribution for two designs is shown in FIG. 10G and FIG. 10H. It can be seen that the magnet configuration in this invention can strengthen the magnetic field significantly over the other existing magnet configuration.

In FIG. 11A and FIG. 11B, we select serval magnet configurations, the remanence of EPMs and IPMs are all set the same. The curve reflecting the relationship between the anchoring force and distance is obtained from the magnetic model calculation after the experimental verification. The resultant force acting on the IPMs immersed in the magnetic field is calculated by:

$${F}_{C} = {\sum }_{a = 1}^{n} {\sum }_{b = 1}^{n} ( {R}_{I} {M}_{Ib} \cdot ∇ ) {B}_{a b}$$

where
M_{Ib}=[m_{Ibx}, m_{Iby}, M_{Ibz}]^{T} ∈ [symbol] ^{3×1} represents the magnetic moment of the b^{th} IPM at the initial position. R_{I}∈ [symbol] ^{3×3} is the rotation matrix of the internal magnetic rotator.
Every force can be further transformed as:

$${F}_{a b} = \frac{3 {μ}_{0}}{4 π {‖ {r}_{a b} ‖}^{4}} ( {R}_{I} {M}_{Ib} {\hat{r}}_{a b}^{T} + {{\hat{r}}_{a b} ( {R}_{I} {M}_{Ib} )}^{T} + ( {\hat{r}}_{a b} \cdot ( {R}_{I} {M}_{Ib} ) ) ( I - 5 {\hat{r}}_{a b} {\hat{r}}_{a b}^{T} ) ) {R}_{E} {M}_{E A}$$

Where r_{ab}=P_{b}−Pₐ is used to denote the space the vector from the a^{th} external magnetic dipole to b^{th} internal magnetic dipole. \hat{r}_{ab} represents the normalized vector of r_{ab}. FIG. 11A gives the result of the anchoring force of different magnet configuration.

Of course, the volume of magnets in different configurations is not all the same, so the unified evaluation index called attractive force per unit volume (FPV) is brought up:

$$F P V = \frac{{F}_{z}}{{V}_{IPMs}}$$

The index includes two most important aspects for clinical application in VATS: a compact dimension of the internal unit that can be inserted through the narrow incision and considerable anchoring force to hold the endoscope on the thick chest wall. The result of this index is shown in FIG. 11B. The comparison results manifest the magnet configuration proposed in this paper can significantly enhance the anchoring performance, the increase of attractive force and FPV is 96.76% and 55.82% averagely.

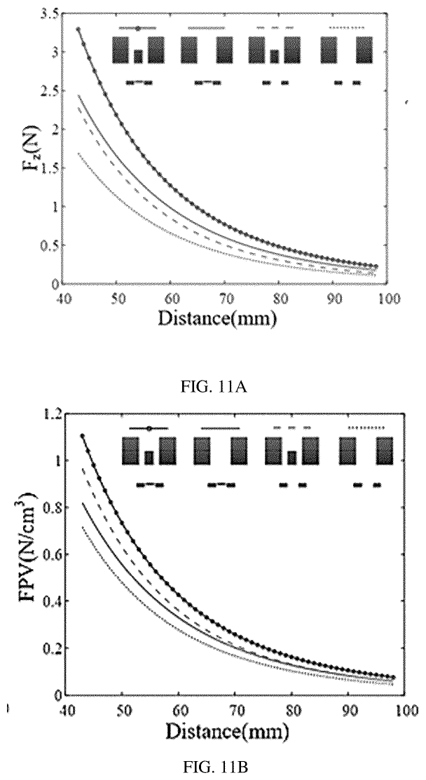

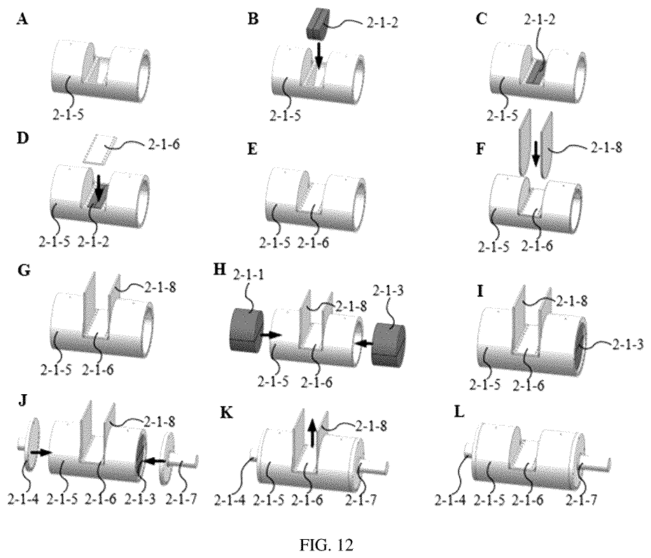

FIG. 12 shows the installation process. During this process, there is a need to reduce the huge repulsive force between the magnets. This invention provides an installation method to overcome this problem:
Step1. Prepare the rotator shell 2-1-5.
Step2. Place the MEPM 2-1-2 in the middle part of the rotator shell 2-1-5.
Step3. Place the middle plate 2-1-6 and fix it on the rotator shell 2-1-5.
Step4. Insert two Permalloy plates for the rotator 2-1-8 on both sides of the MEPM 2-1-2 to shield magnetic field.
Step5. Install the LEPM 2-1-1 and REPM 2-1-3.
Step6. Place the left plate 2-1-4 and right plate 2-1-7 and fix them on the rotator shell 2-1-5.
Step7. Remove the two Permalloy plates for the rotator 2-1-8.

As for the working principle of the system, utilizing the magnetic coupling between the EPMs and IPMs, the configuration can realize the rotation motion and translation motion under the premise of anchoring the capsule against the inner wall of the body cavity. The rotation of the internal unit includes the tilt motion and pan motion, and are achieved by the torques applied by EPMs around the corresponding axis. The tilt torque and pan torque acting on the capsule is generated by rotating the external unit along the corresponding axis to change the EPMs' magnetic field coupled to the IPMs. The translation motion of the internal endoscope is on the curved surface of the chest wall, requiring the magnetic force along the corresponding axis. The movement of the external unit above the patient's body cavity can produce the translational force.

### Manufacturing Method

For the magnets, they can be permanent magnet, electromagnet, magnetized soft materials and so on. They can be made in the lab, bought from the store or produced by factory. For the shell, plate, and frame, they can be fabricated with the material with high strength and connected with the method with high stability.

### Reference List

1. U.S. Pat. No. 6,902,528 B1. Method and apparatus for magnetically controlling catheters in body lumens and cavities, 18 Feb. 2003.
2. J. Cadeddu, R. Fernandez, M. Desai, R. Bergs, C. Tracy, S.-J. Tang, P. Rao, M. Desai, and D. Scott, “Novel magnetically guided intraabdominal camera to facilitate laparoendoscopic single-site surgery: initial human experience,” Surgical endoscopy, vol. 23, no. 8, pp. 1894-1899, 2009.
3. M. Simi, M. Silvestri, C. Cavallotti, M. Vatteroni, P. Valdastri, A. Menciassi, and P. Dario, “Magnetically activated stereoscopic vision system for laparoendoscopic single-site surgery,” IEEE/ASME Transactions on Mechatronics, vol. 18, no. 3, pp. 1140-1151, 2012.
4. M. Simi, R. Pickens, A. Menciassi, S. D. Herrell, and P. Valdastri, “Fine tilt tuning of a laparoscopic camera by local magnetic actuation: Two-port nephrectomy experience on human cadavers,” Surgical innovation, vol. 20, no. 4, pp. 385-394, 2013.
5. N. Garbin, P. R. Slawinski, G. Aiello, C. Karraz, and P. Valdastri, “Laparoscopic camera based on an orthogonal magnet arrangement,” IEEE Robotics and Automation Letters, vol. 1, no. 2, pp. 924-929, 2016.
6. X. Liu, G. J. Mancini, and J. Tan, “Design of a unified active locomotion mechanism for a wireless laparoscopic camera system,” in 2014 IEEE/RSJ International Conference on Intelligent Robots and Systems. IEEE, 2014, pp. 1294-1301
7. X. Liu, G. J. Mancini, and J. Tan, “Design of a unified active locomotion mechanism for a capsule-shaped laparoscopic camera system,” in 2014 IEEE International Conference on Robotics and Automation (ICRA). IEEE, 2014, pp. 2449-2456.
8. H. Wei, W. Tan, K. Li, and D. Xu, “Design and modeling of a magnetically actuated laparoscopic robot,” in 2019 14th IEEE Conference on Industrial Electronics and Applications (ICIEA). IEEE, 2019, pp. 149-154.
9. T. Cheng, C. S. H. Ng, P. W. Y. Chiu, and Z. Li, “Design and prototyping of a soft magnetic anchored and guidance endoscope system,” in 2017 IEEE/RSJ International Conference on Intelligent Robots and Systems (IROS). IEEE, 2017, pp. 2902-2908.
10. T. Cheng, X. Zhang, C. S. H. Ng, P. W. Y. Chiu, and Z. Li, “A novel magnetic anchored and steered camera robot for single port access surgery,” in 2018 IEEE International Conference on Robotics and Automation (ICRA). IEEE, 2018, pp. 1406-1412.
11. X. Liu, G. J. Mancini, Y. Guan, and J. Tan, “Design of a magnetic actuated fully insertable robotic camera system for single-incision laparoscopic surgery,” IEEE/ASME Transactions on Mechatronics, vol. 21, no. 4, pp. 1966-1976, 2015.
12. T. Cheng, W. Li, C. S. H. Ng, P. W. Y. Chiu, and Z. Li, “Visual servo control of a novel magnetic actuated endoscope for uniportal video assisted thoracic surgery,” IEEE Robotics and Automation Letters, vol. 4, no. 3, pp. 3098-3105, 2019.
13. T. Cheng, W. Li, W. Y. Ng, Y. Huang, J. Li, C. S. H. Ng, P. W. Y. Chiu, and Z. Li, “Deep learning assisted robotic magnetic anchored and guided endoscope for real-time instrument tracking,” IEEE Robotics and Automation Letters, vol. 6, no. 2, pp. 3979-3986, 2021.
14. U.S. Pat. No. 8,084,898 B2. Magnetic actuator, magnetic actuator operating method, and capsule endoscope using the same, 27 Dec. 2011.
15. U.S. Pat. No. 11,033,183 B2. Wireless magnetically steerable endoscope, 15 Jun. 2021.

What is claimed is:

1. A magnetic anchored and actuated system, comprising:
a. An internal unit for insertion into a patient's body, comprising an internal frame, an internal magnetic rotator, and a function module; and
b. An external unit for anchoring and controlling locomotion of said internal unit, comprising an external frame, an external magnetic rotator, and an actuation module;
wherein,
said internal magnetic rotator comprises an internal shell, a left internal magnetic component, a middle internal magnetic component, and a right internal magnetic component;
said external magnetic rotator comprises an external shell, a left external magnetic component, a middle external magnetic component, and a right external magnetic component;
said left external magnetic component and said left internal magnetic component are radially magnetized with magnetization direction of said left external magnetic component being same as said left internal magnetic component;
said right external magnetic component and said right internal magnetic component are radially magnetized with magnetization direction of said right external magnetic component being same as said right internal magnetic component;
each of said left external magnetic component and said left internal magnetic component has a magnetization direction opposite to each of said right external magnetic component and right internal magnetic component;
said middle external magnetic component and said middle internal magnetic component are axially magnetized with magnetization direction of said middle external magnetic component being opposite to said middle internal magnetic component; and
each of said left internal magnetic component, middle internal magnetic component and right internal magnetic component is magnetically coupled to each of said left external magnetic component, middle external magnetic component, and right external magnetic component respectively;
said actuation module controls motion of said external magnetic rotator, said motion comprises rotation and translation; and
said function module provides one or more specific functionalities along with movement of said internal magnetic rotator.

2. The system of claim 1, wherein said left external magnetic component, middle external magnetic component, right external magnetic component, left internal magnetic component, middle internal magnetic component or right internal magnetic component comprises one or more magnets.

3. The system of claim 2, wherein said one or more magnets are selected from the group consisting of permanent magnets, electromagnets and soft magnetic materials.

4. The system of claim 1, wherein said left internal magnetic component, right internal magnetic component, left external magnetic component, right external magnetic component, middle internal magnetic component or middle external magnetic component has a shape selected from the group consisting of cylindrical, semi-cylindrical, cuboid, sphere, semi-sphere, and ellipsoid.

5. The system of claim 1, wherein said internal shell holds in place said left internal magnetic component, said middle internal magnetic component and said right internal magnetic component.

6. The system of claim 1, wherein said internal magnetic rotator further comprises one or more onboard sensors for measuring location and motion.

7. The system of claim 6, wherein said one or more onboard sensors are selected from the group consisting of inertial measurement unit, gyroscope, accelerator, hall sensor, and optical marker.

8. The system of claim 1, wherein said internal magnetic rotator is connected via a first connection mechanism to said internal frame for rotation around a central axis.

9. The system of claim 8, wherein said first connection mechanism comprises one or more selected from the group consisting of bearings, shaft, compliant structure, and soft structure.

10. The system of claim 1, wherein said one or more specific functionalities comprises providing one or more functions selected the group consisting of lighting, imaging, tissue/organ retraction, instrument manipulation, and fluid suction.

11. The system of claim 1, wherein said function module is attached to the internal magnetic rotator by one or more methods selected from the group consisting of glue, movable joints, and compliant/soft structures.

12. The system of claim 1, wherein said external shell comprises a shell body, a left plate, a middle plate and a right plate for holding said left external magnetic component, middle external magnetic component and right external magnetic component respectively; wherein said shell body, left plate and right plate are made of non-ferromagnetic materials.

13. The system of claim 1, wherein said external magnetic rotator further comprises one or more onboard sensors for measuring location and motion.

14. The system of claim 13, wherein said one or more onboard sensors are selected from the group consisting of inertial measurement unit, gyroscope, accelerator, hall sensor, encoder, and optical marker.

15. The system of claim 1, wherein said external magnetic rotator is connected via a second connection mechanism to said external frame for rotation around a central axis.

16. The system of claim 15, wherein said second connection mechanism comprises one or more selected from the group consisting of bearings, shaft, compliant structure, and soft structure.

17. The system of claim 1, wherein said actuation module comprises an actuator for rotating said external magnetic rotator and an external holding device for spatially moving said external magnetic rotator.

18. The system of claim 17, wherein said actuator comprises a motor.

19. The system of claim 17, wherein said external holding device comprises a robot arm.

20. A method for assembling the external magnetic rotator in the system of claim 1, said method comprises the steps of:
a. providing a shell body made of non-ferromagnetic materials;
b. Placing the middle external magnetic component in middle part of said shell body;
c. Placing and fixing a middle plate;
d. Inserting two magnetic shielding plates on both sides of the middle external magnetic component to shield magnetic field;
e. Installing the left external magnetic component and the right external magnetic component;
f. Placing and fixing a right plate and a left plate, wherein said left plate and right plate are made of non-ferromagnetic materials; and
g. Remove the two magnetic shielding plates.

21. The method of claim 20, wherein said magnetic shielding plates are made of permalloy.
